(19) United States
(12) Patent Application Publication
(10) Pub. No.: US 2025/0179504 A1
(43) Pub. Date: Jun. 5, 2025

(54) Biosynthesis Of Apocarotenoids By Controlling Oxidative Stress

(71) Applicants: Agency for Science, Technology and Research, Singapore (SG); Fermatics Pte. Ltd., Singapore (SG)

(72) Inventors: Ching-Ning Huang, Singapore (SG); Xixian Chen, Singapore (SG); Congqiang Zhang, Singapore (SG)

(21) Appl. No.: 18/843,261

(22) PCT Filed: Mar. 1, 2023
(86) PCT No.: PCT/SG2023/050123

§ 371 Date: Sep. 1, 2024

(30) Foreign Application Priority Data
Mar. 1, 2022 (SG) 10202202053S

(51) Int. Cl. C12N 15/52; C12N 9/02; C12N 9/08; C12N 9/10; C12N 9/90; C12P 7/00

(52) U.S. Cl. CPC C12N 15/52; C12N 9/0065; C12N 9/0069; C12N 9/1085; C12N 9/90; C12P 7/00; C12Y 111/01006; C12Y 113/11069; C12Y 205/0101; C12Y 205/01029; C12Y 505/01019

(57) ABSTRACT

The present invention relates to host cells comprising genes of the mevalonate, lycopene and α-ionone pathways and the hydroperoxide reductase or catalase genes. The present invention also relates to methods of producing apocarotenoids as well as kits for producing apocarotenoids comprising the host cells.

### CROSS-REFERENCE TO RELATED APPLICATIONS

This application claims the benefit of priority of Singapore Application No. 10202202053S, filed 1 Mar. 2022, the contents of it being hereby incorporated by reference in its entirety for all purposes.

### FIELD OF THE INVENTION

The invention is in the field of biotechnology. The invention relates to improved methods for producing apocarotenoids. In particular, the invention relates to the biosynthesis of α-ionone using genetically modified host organisms.

### SEQUENCE LISTING

The instant application contains a Sequence Listing which has been submitted electronically in XML format and is hereby incorporated by reference in its entirety. Said XML copy, created 1 Mar. 2023, is named 78696SG2_Sequence Listing.xml and is 20 kilobytes in size.

### BACKGROUND OF THE INVENTION

Apocarotenoids are a class of compounds which are generated through oxidative cleavage of carotenoids. Apocarotenoids are widely distributed in bacteria, fungi, plants and animals. The apocarotenoids have diverse biological functions and act as precursors of homones (abscisic acid, strigolactones), pigments, signalling compounds and aromatic compounds. These carotenoid-derived aromatic compounds comprise ionones (α- and β-ionone) which are a group of natural ketone compounds composed of 13 carbons with a monocyclic terpenoid backbone, and exist in various flowers and fruit including rose, sweet osmanthus, orris root, and raspberry. α- and β-ionone are produced through the oxidation of carotenoids such α-carotene or ε-carotene by carotenoid cleavage oxygenases. In particular, α-ionone has a violet-like aroma, a raspberry- and blackberry-flavor, and an extremely low odor threshold from 0.4 to 3.2 ppb (parts per billion). These properties of α-ionone make them widely applied in the flavour and fragrance industry.

With its high commercial value and demand, the main production of commercial α-ionone is through chemical synthesis as extraction from natural materials is not viable on a large scale due to extremely low abundance.

Therefore, there is a need to improve existing methods of apocarotenoids biosynthesis that produces higher yields.

### SUMMARY

In one aspect, there is provided a host cell comprising one or more vectors comprising a polynucleotide sequence encoding:
one or more genes of the mevalonate pathway;
one or more genes of the lycopene pathway;
one or more genes of the α-ionone pathway; and
one or more hydroperoxide reductase or catalase genes.

In another aspect, there is provided a method of producing one or more apocarotenoids comprising culturing the host cell as described herein in a culture medium.

In another aspect, there is provided a kit for producing one or more apocarotenoids, wherein the kit comprises the host cell as described herein with instructions for use.

### Definitions

As used herein, the term “carotenoid” refers to a class of naturally occurring pigments synthesized by plants, animals, algae and microbes. Carotenoids have structures of an electron-rich polyene chain with nine or more conjugated bonds and possess photo-protection, light-harvesting and anti-oxidant properties.

As used herein, the term “apocarotenoid” refers to a class of naturally occurring compounds that are derived from carotenoids through oxidative cleavage that are catalyzed by enzymes. These enzymes are carotenoid cleavage dioxygenases (CCDs).

As used herein, the term “variant” refers to a modification in the DNA sequence. The modification in the DNA sequence includes mutation, truncation, translocation, substitution, deletion and insertion, resulting in the alteration of the activity of the gene.

The region of the DNA is typically located near the transcription start site of a gene and upstream on the DNA. A promoter may be inducible or non-inducible. The term “inducible promoter” as used herein refers to a promoter that can be regulated in the response to specific stimuli, also known as inducers. The promoter system may be modified to be inducible. Examples of inducible promoter systems in include the Tet-on system, Tet-off system, T7 system, Trp system, Tac system, lambda cl857-PL system, bacterial EL222 system and Lac system. A promoter may also be a constitutive promoter which is a promoter that is always active.

As used herein, the term “overexpressed” in the context of a gene or a protein refers to an increase in expression of a gene or a protein. Overexpression may also refer to the expression of a gene in a scenario where the gene would otherwise not be expressed. It would generally be understood that the overexpression of a gene or a protein is relative to a baseline expression of the gene or the protein. The baseline expression of a gene or a protein would be understood to mean the expression level of an unmutated gene or a wild type gene, or in the context where the gene would otherwise not be expressed, the expression level of the background/noise. In this context, the ahpC/F or the KatG is overexpressed and it would be understood that the expression of ahpC/F and KatG is increased relative to basal level of ahpC/F and KatG in the host cell (i.e. additional copies of ahpC/F and KatG).

As used herein, the term “about”, in the context of concentrations of components and percentages of compounds, typically refers to +/−5% of the stated value, +/−4% of the stated value, more typically +/−3% of the stated value, more typically, +/−2% of the stated value, even more typically +/−1% of the stated value, and even more typically +/−0.5% of the stated value. Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### BRIEF DESCRIPTION OF THE DRAWINGS

The invention will be better understood with reference to the detailed description when considered in conjunction with the non-limiting examples and the accompanying drawings, in which:

FIG. 1 shows a schematic diagram of the biosynthetic pathway of α-ionone. This pathway contains 4 modules: Mevalonate pathway (module 1 and 2), Lycopene pathway (module 3) and α-ionone pathway (module 4). The genes expressed encode the following enzymes: atoB, Acetoacetyl-CoA thiolase; hmgS, HMG-COA synthase; thmgR, truncated HMG-CoA reductase; mevk, mevalonate kinase; pmk, phosphomevalonate kinase; pmd, mevalonate pyrophosphate decarboxylase; idi, IPP isomerase; ispA, FPP synthase; crtE, GGPP synthase; crtB, phytoene synthase; crtI, lycopene-beta-cyclase; IcyE, lycopene-epsilon-cyclase. Abbreviation for the compounds: CCD1, carotenoid cleavage dioxygenase 1; HMG-COA, 3-hydroxy-3-methyl-glutaryl-coenzyme A; MVA, mevalonate; MVAP, phosphomevalonate; MVAPP, diphosphomevalonate; IPP, isopentenyl pyrophosphate; DMAPP, dimethylallyl pyrophosphate; GPP, geranyl pyrophosphate; FPP, farnesyl pyrophosphate; GGPP, geranylgeranyl pyrophosphate. Dashed arrow indicates multiple enzymatic steps.

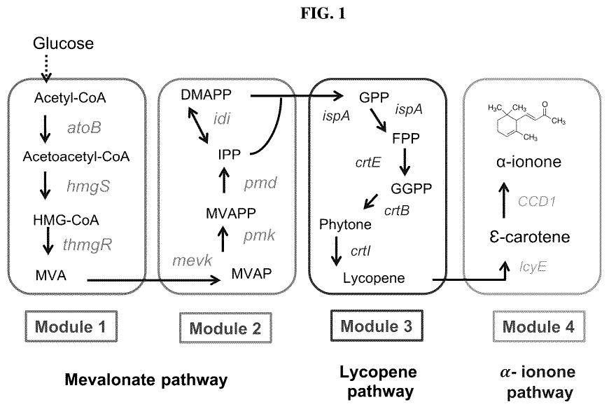

FIG. 2 shows that the titer, content and optical density of α-ionone production by different strains. FIG. 2A shows AI_2211, A: 1 ml defined medium+200 μl Dodecane, 300 rpm (83.55 mg/l) by Snape tube. B: 10 ml defined medium+10 ml Dodecane, 100 rpm (39.15 mg/l) by the flask. C: 10 ml defined medium+10 ml Dodecane, 300 rpm (19.25 mg/l) by the flask. FIG. 2B shows AI_2211, AI_3211, AI_2217 and AI_2218, in 10 ml defined medium+10 ml Dodecane by flask under 100 rpm and 300 rpm. The data is an average of duplicate data.

FIG. 3 shows the ROS-Glo™ Assay signals from H₂O₂ production by AI_2211 and AI_2218 under 250 rpm or 500 rpm by RTS. After incubation for 1.5 hr, 100 μl of each testing of AI_2211 and AI_2218 mixed culture plated in a 96-well white cell culture plate and 100 μl of ROS-Glo™ Detection Solution was added to the wells. Luminescence was determined with a GloMax® Multi+Luminometer. The average relative light unit (RLU) and standard deviation of quadruplicate samples were calculated.

FIG. 4 shows the ratio of MHO/α-ionone Production of AI_2211 and AI_2218 under 100 rpm and 300 rpm by flask with cell pellet. The fold of AI_2211 100 rpm (0.24), 300 rpm (0.48) and AI_2218 100 rpm (0.26), 300 rpm (0.22). The color of the pellets of AI_2211 and AI_2218, 100 rpm shows orange; AI_2211, 300 rpm shows colorless and AI_2218, 300 rpm is yellow.

FIG. 5 shows the time course profiles for ionones and optical density (OD₆₀₀) produced using a bio-reactor of AI_2218. The end point is 127 hr and OD₆₀₀ is 229.3, and for the titer of α-ionone is 679.9 mg/l, ß-ionone is 35.2 mg/l and psi-ionone is 105.8 mg/l.

FIG. 6 depicts a proposed scheme illustrating how H₂O₂ affects α-ionone production. (i) The pathway without oxidative stress. The H₂O₂ production is low due to low shaking speed. (ii) The pathway with oxidative stress. The H₂O₂ is high due to high shaking speed. H₂O₂ will degrade lycopene, (a substrates of CCD1), to MHO and potentially inactivate CCD1 (an enzyme containing a key Fe²⁺ atom in the active site that can be oxidized to Fe³⁺ by H₂O₂) to further reduce the pathway flux towards α-ionone production. (iii) The pathway without oxidative stress due to H₂O₂ elimination by AhpC/F. Lycopene will not be degraded to MHO and CCD1 will not be inactivated which will further increase α-ionone production.

FIG. 7 shows the α-ionone producing strains, AI_2211 and AI_2218. AI_2211, Module 1: Tm2-AHT+CCD1, Module 2: Tm3-MPPI, Module 3: Tm1 BIA+MbGGPPs and Module 4: Tm1-LTOM. AI_2218, Module 1: Tm2+CCD1, Module 2: Tm3-MPPI, Module 3: Tm1-MbGGPPs, Module 4: Tm1-LTOM-ahpC/F.

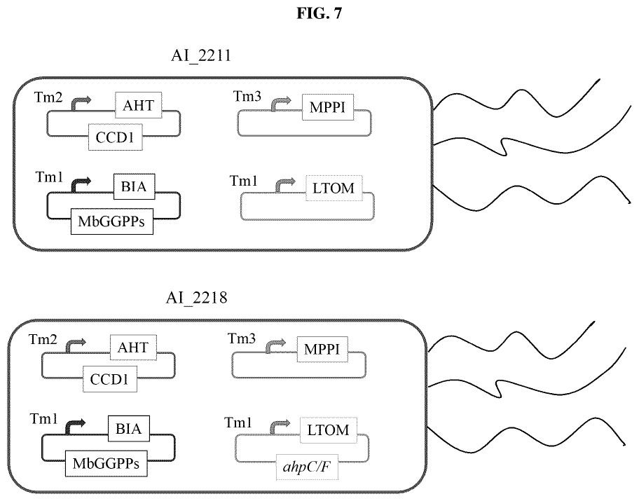

FIG. 8 shows the comparison of α-ionone titer by different strains: AI_0000, AI_2211 and AI_2218. AI_0000 (24.1 mg/l), AI_2211 (13 mg/l) and AI_2218 (38.4 mg/l). All did in 10 ml defined medium+10 ml Dodecane by flask under 300 rpm shaking speed. The data is an average of duplicate data.

FIG. 9 depicts the effect of H₂O₂ on LcyE activity leading to lycopene accumulation (i) The pathway without oxidative stress. The H₂O₂ production is low due to low shaking speed. (ii) H₂O₂ could oxidize the reduced FAD of LcyE and inactivate the enzyme. Lycopene would then accumulate and be cleaved to MHO by CCD1 and potentially inactivate CCD1 (an enzyme containing a key Fe²⁺ atom in the active site that can be oxidized to Fe³⁺ by H₂O₂) to further reduce the pathway flux towards α-ionone production. (iii) The pathway without oxidative stress due to H₂O₂ elimination by AhpC/F. Lycopene will not be degraded to MHO and CCD1 will not be inactivated which will further increase α-ionone production.

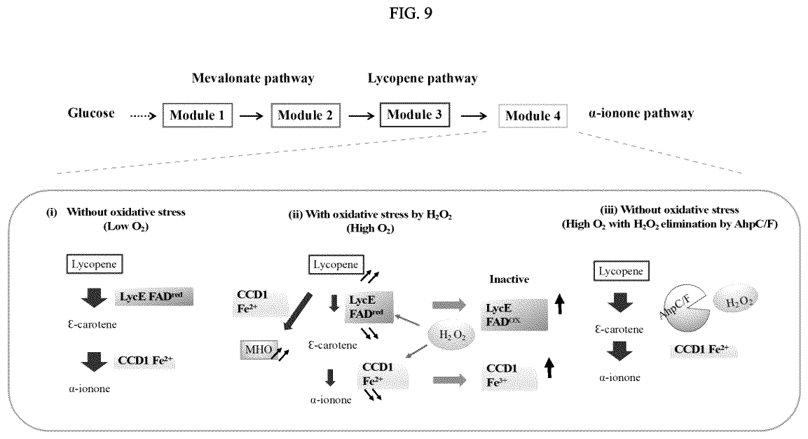

FIG. 10 shows α-ionone production by AI_2211 (Tm2+CCD1/Tm3-MPPI/Tm1-MbGGPPs/Tm1-LTOM) & AI_3211 (Tm2-AHT/Tm3-MPPI/Tm1-MbGGPPs/Tm1-LTOM). AI_2211, 100 rpm (42 mg/l), 300 rpm (11 mg/l). AI_3211, 100 rpm (17 mg/l), 300 rpm (25 mg/l). Both were performed in 10 ml defined medium+10 ml Dodecane by flask. The data is an average of duplicate data.

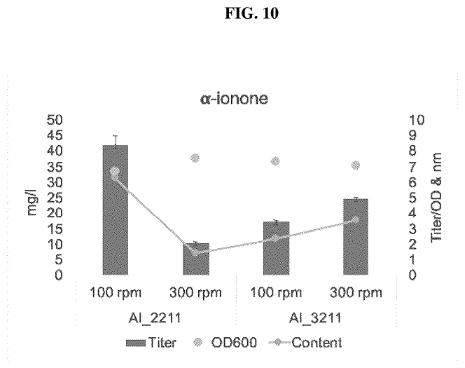

FIG. 11 shows α-ionone production by strains overexpressing catalase genes: catalase G (katG), alkyl hydroperoxide reductase (ahpC/F). AI_2217 (Tm2+CCD1/Tm3-MPPI/Tm1-MbGGPPs/Tm1-LTOM-katG) AI_2218 (Tm2+CCD1/Tm3-MPPI/Tm1-MbGGPPs/Tm1-LTOM-ahpC/F AI_2217, 100 rpm (35 mg/l), 300 rpm (21 mg/l). AI_2218, 100 rpm (27 mg/l), 300 rpm (38 mg/l). Both were performed in 10 ml defined medium+10 ml Dodecane by flask. The data is an average of duplicate data.

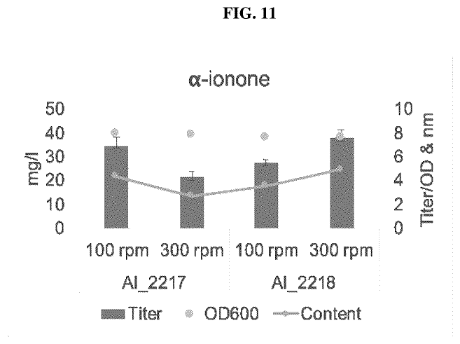

FIG. 12 shows α-ionone titer by different strains: AI_2211, AI_3211, AI_2217 and AI_2218. AI_2211, 100 rpm (49 mg/l), 300 rpm (14 mg/l). AI_3211, 100 rpm (29 mg/l), 300 rpm (35 mg/l). AI_2217, 100 rpm (45 mg/l), 300 rpm (24 mg/l). AI_2218, 100 rpm (39 mg/l), 300 rpm (42 mg/l). All were performed in 10 ml defined medium+10 ml Dodecane by flask. The data is an average of duplicate data.

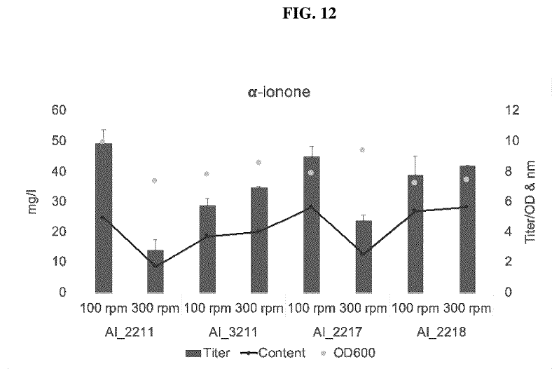

### DETAILED DESCRIPTION OF THE PRESENT INVENTION

In a first aspect, the present invention refers to host cell comprising one or more vectors comprising a polynucleotide sequence encoding:
one or more genes of the mevalonate pathway;
one or more genes of the lycopene pathway;
one or more genes of the α-ionone pathway; and
one or more hydroperoxide reductase or catalase genes.

The host cell may comprise any number of vectors that allows expression of the one or more genes of the mevalonate, lycopene, α-ionone pathways and hydroperoxide reductase or catalase genes. For example, the host cell may comprise one vector, two vectors, three vectors, four vectors, five vectors, six vectors or seven vectors. It will be appreciated by a person skilled in the art that the one or more genes of the mevalonate pathway, the one or more genes of the lycopene pathway, the one of more genes of the α-ionone pathway; and the one or more hydroperoxide reductase or catalase genes may be located on one or more vectors in different combinations. In one example, the one or more genes of the mevalonate pathway may be located one vector, the one or more genes of the lycopene pathway may be located on another vector, the one or more genes of the α-ionone pathway may be located on another vector and the one or more hydroperoxide reductase or catalase genes be located on yet another vector. In another example, the one or more genes of the mevalonate pathway and the one or more genes of the lycopene pathway may be located on one vector, the one or more genes of the lycopene pathway and the α-ionone pathway may be located on another vector, the one or more genes of the mevalonate pathway and the α-ionone pathway may be located on another vector, and the one or more genes of the lycopene pathway and the one or more hydroperoxide reductase or catalase genes may be located on yet another vector. In yet another example, the one or more genes of the mevalonate pathway and the α-ionone pathway may be located on one vector, the one or more genes of the mevalonate pathway and the α-ionone pathway may be located on another vector, the one or more genes of the lycopene pathway may be located on another vector and the one or more hydroperoxide reductase or catalase genes may be located on yet another vector. In yet another example, the one or more genes of the mevalonate pathway may be located on two vectors, the one or more genes of the lycopene pathway may be located on one vector and the one or more hydroperoxide reductase or catalase genes may be located on yet another vector. It is to be understood that the above examples are not exhaustive and are merely meant to illustrate that the genes may be located on one or more vectors in various combinations.

In one example, the host cell comprises four vectors. In one example, the host cell comprises:
a) a first vector comprising the polynucleotide sequence encoding one or more genes of the mevalonate pathway;
b) a second vector comprising the polynucleotide sequence encoding one or more genes of the mevalonate pathway;
c) a third vector comprising the polynucleotide sequence encoding one or more genes of the lycopene pathway; and
d) a fourth vector comprising the polynucleotide sequence encoding one or more genes of the α-ionone pathway and the polynucleotide sequence encoding one or more hydroperoxide reductase or catalase genes.

In one example, the one or more genes of the mevalonate pathway may include but not limited to hmgS, atoB, hmgR, mevk, pmk, pmd and idi. In one example, the hmgR gene is truncated.

In one example, the one or more genes of the lycopene pathway may include but not limited to crtBI, crtB, crtI, a farnesyl diphosphate synthase gene and a geranylgeranyl diphosphate synthase (GGPPs) gene.

In one example, the GGPPs gene is isolated from a prokaryote. In one example, the prokaryote is archaea. The archaea may be Methanocaldococcus jannaschii or Methanococcus maripaludis. In another example, the prokaryote may be bacteria. The bacteria may be Methanobacterium or Deinococcus radioduran. In a preferred example, the bacterium is Methanobacterium.

In one example, the one or more genes of the α-ionone pathway may include but is not limited to a lycopene cyclase gene and a CCD gene.

In addition, to the one or more genes of the α-ionone pathway, the host cell may further comprise polynucleotide sequences encoding one or more additional CCD genes. The additional CCD genes may be located on the first, second, third, fourth vectors, or combinations thereof. Therefore, the host cell may comprise a total of one, two, three, four or more copies of the CCD gene.

In one example, the host cell comprises one additional copy of the CCD gene wherein the polynucleotide sequence encoding the additional CCD gene is located on a vector comprising the one or more genes of the mevalonate pathway.

In one example, the host cell comprises two additional copies of the CCD gene wherein the polynucleotide sequences encoding the two additional CCD genes are located on a vector comprising the one or more genes of the α-ionone pathway.

In another example, the host cell comprises two additional copies of the CCD gene wherein the polynucleotide sequences encoding the two additional CCD genes of the α-ionone pathway are located on one vector comprising the one or more genes of the mevalonate pathway and on another vector comprising the one or more genes of the lycopene pathway and the α-ionone pathway. In yet another example, the host cell comprises three additional copies of the CCD genes wherein the polynucleotide sequences encoding the three additional CCD genes are located on one vector comprising one or more genes of the mevalonate pathway, on another vector comprising the one or more genes of the lycopene pathway and on yet another vector comprising the one or more genes of the α-ionone pathway. In yet another example, the host cell comprises four additional copies of the CCD gene wherein the polynucleotide sequences encoding the four additional CCD1 genes of the α-ionone pathway are located on two vectors comprising the one or more genes of the mevalonate pathway and on one vector comprising the one or more genes of the α-ionone pathway and the one or more hydroperoxide reductase or catalase genes. In yet another example, the host cell comprises four additional copies of the CCD gene wherein the polynucleotide sequences encoding four additional CCD1 genes of the α-ionone pathway are located on one vector comprising the one or more genes of the lycopene pathway and on another vector comprising the one or more genes of the α-ionone pathway and the one or more hydroperoxide reductase or catalase genes. It is to be understood that these examples are not exhaustive and the one or more additional copies of the CCD genes can be located on any vector or combinations thereof.

In one example, the CCD gene may be a CCD1 or a CCD4 gene. In one preferred example, the CCD gene is CCD1 gene.

In one example, the one or more CCD1 genes is isolated from a plant. The plant may comprise Osmanthus fragrans, Arabidopsis thaliana, Vitis vinifera and Petunia hybrid. In a preferred example, the one or more CCD1 genes is isolated from Osmanthus fragrans (OfCCD1). The one or more OfCCD1 genes comprises the polypeptide sequence set forth in SEQ ID NO: 1.

In one example, the one or more of the OfCCD1 genes is mutated at the loop of the active site. The OfCCD gene may be mutated at one or more amino acid positions at the loop of the active site. The OfCCD gene may be mutated at one amino acid position, two amino acid positions, three amino acid positions, four amino acid positions, five amino acid positions, six amino acid positions at the loop of the active site. In a preferred example, the mutation of the OfCCD1 is at three amino acid positions at the loop of the active site. The mutations comprise a substitution of lysine at position 425 with serine, a substitution of aspartate at position number 424 with asparagine and a substitution of lysine at position number 428 with alanine. The mutated OfCCD1 gene may comprise the polynucleotide sequence set forth in SEQ ID NO: 2.

In one example, the one or more of the OfCCD1 is truncated.

The one or more CCD genes may be fused at the N-terminal with a protein or peptide (fusion partner). It would generally be understood that fusion partner refers to a protein or a peptide that is fused with another protein or peptide. In one example, the N-terminal fusion partner may include but not limited to small ubiquitin-like modifier protein, maltose binding protein and thioredoxin (trxA). In a preferred example, the N-terminal fusion partner is trxA. In one example, the OfCCD1 is fused to trxA.

In one example, the one or more of the OfCCD1 and trxA genes are overexpressed. The OfCCD1 gene may be expressed with the trxA gene and in one example, the trxA gene may be overexpressed with the OfCCD1 gene when fused to the trxA gene.

When the host cell comprises more than one copy of the CCD gene, each copy of the CCD gene may be a wild type CCD gene or a mutant of the CCD gene or combinations thereof. The polynucleotide sequences encoding one or more CCD genes may be located on one or more vectors. In one example, the vector comprising one or more genes of the mevalonate pathway comprises the polynucleotide sequence of a wild type OfCCD1 gene fused with TrxA and the vector comprising one or more genes of the α-ionone pathway and one or more hydroperoxide reductase or catalase genes comprises the polynucleotide sequence of a mutated OfCCD1 gene fused with TrxA. In another example, the vector comprising one or more genes of the mevalonate pathway comprises the polynucleotide sequence of a wild type CCD4 gene and the vector comprising one or more genes of the lycopene pathway comprises the polynucleotide sequence of a wild type OfCCD1 gene. In another example, the vector comprising one or more genes of the lycopene pathway comprises the polynucleotide sequence of a wild type OfCCD1 gene and the vector comprising one or more genes of the lycopene pathway comprises the polynucleotide sequence of a mutated OfCCD1 gene. In yet another example, the vector comprising one or more genes of the mevalonate pathway comprises the polynucleotide sequence of a mutated OfCCD1 gene and the vector comprising one or more genes of the lycopene pathway comprises the polynucleotide sequence of a truncated OfCCD1. In yet another example, the vector comprising one or more genes of the mevalonate pathway comprises the polynucleotide sequence of a wild type OfCCD1 gene, the vector comprising one or more genes of the lycopene pathway comprises the polynucleotide sequence of a mutated OfCCD1 and the vector comprising one or more genes of the α-ionone pathway and one or more hydroperoxide reductase or catalase genes comprises the polynucleotide sequence of a CCD4 gene. It is to be understood that these examples are not exhaustive and the one or more vectors in the host cell may comprise any of the CCD genes as described herein.

In one example, the lycopene cyclase gene is a lycopene epsilon-cyclase (LCYe) gene. In one example, the LCYe gene is isolated from a plant including but not limiting to Lactuca sativa, Arabidopsis thaliana, Nicotiana tabacum and Brassica oleracea var. capitata. In a preferred example, the LCYe is isolated from Lactuca sativa.

The LCYe gene comprises one or more mutations, resulting in the truncation of the LCYe protein. In one example, the LCYe gene is truncated at the N-terminal. In one example, the LCYe is truncated from amino acid positions 1 to 50. In one example, the truncated LCYe comprise the polynucleotide sequence set forth in SEQ ID NO: 3.

In one example, the one or more hydroperoxide reductase or the catalase gene is isolated from a prokaryote. In one preferred example, the prokaryote is Escherichia coli.

In one example, the hydroperoxide reductase gene isolated from Escherichia coli is alkyl hydroperoxide reductase (ahpC/F). In another example, the catalase gene isolated from Escherichia coli is catalase G (KatG).

In one example, the ahpC/F and/or the KatG is overexpressed. Overexpression may be achieved by various means that would be generally known in the art. In one example, overexpression may be achieved by expression of more than one copy of the ahpC/F and/or the KatG gene in the host cell. In some examples, the host cell may express one copy of ahpC/F and/or KatG endogenously in the genome of the host cell and one or more additional copies in the genome or on one or more vectors.

In one example, the polynucleotide sequence encoding the one or more genes in the one or more vectors would be understood to be operably linked to one or more inducible promoters. It would be generally understood that any promoter that allows the expression of the polynucleotide sequence may be employed. Examples of the promoters include but are not limited to T7 RNA polymerase promoter, the lac promoter, araBAD promoter, tac promoter, lambda cl857-PL promoter and the T5 promoter.

In some examples, the promoter may be an inducible promoter. In one example, the promoter may be naturally inducible. In one example, the promoter may be engineered to be inducible. It will be appreciated that any suitable inducible promoter system may be used. Inducible promoter systems may be induced by an inducer or stimuli including but not limited to chemical inducers, light or heat.

In one example, the polynucleotide sequence is operably linked to an inducible promoter in one or more vectors in one or more vectors and operably linked to an uninducible promoter in other vectors. For example, the polynucleotide sequence encoding the one or more genes of the mevalonate pathway and the lycopene pathway is operably linked to an inducible promoter in two vectors and the polynucleotide sequence encoding one or more genes of the α-ionone pathway and the one or more hydroperoxide reductase or catalase genes is operably linked to an uninducible promoter in the two vectors. In another example, the polynucleotide sequence encoding the one or more genes of the mevalonate pathway, the lycopene pathway, the α-ionone pathway and the one or more hydroperoxide reductase or catalase genes is operably linked to an inducible promoter in four vectors. In yet another example, the polynucleotide sequence encoding the one or more genes of the mevalonate pathway is operably linked to an inducible promoter in one vector and the polynucleotide sequence encoding the one or more genes of the lycopene pathway, the α-ionone pathway and the one or more hydroperoxide reductase or catalase genes operably linked to an uninducible promoter in two vectors.

In one example, the polynucleotide sequence encoding one of more genes is operably linked to one or more inducible promoter. In one example, the inducible promoter is a T7 promoter or a variant of the wild-type T7 promoter. The variant of the wild-type T7 promoter may be generated via mutations to the wild-type promoter. In one example, the T7 promoter variant may include but not limited to TM1, TM2 and TM3. It will generally be understood that each of the polynucleotide sequence comprising one or more genes of the mevalonate pathway, the lycopene pathway, the α-ionone pathway and the one or more hydroperoxide reductase or catalase genes can be operably linked to an inducible promotor and different combinations of the inducible promoters may be used with each of the vectors of the invention. For example, the inducible promoter in the vector comprising the one or more genes of mevalonate pathway is TM2, the inducible promoter in another vector comprising the one or more genes of the mevalonate pathway is TM3, the inducible promoter in another vector comprising the one or more genes of the lycopene pathway is TM1 and the inducible promoter in another vector comprising the one or genes of the α-ionone pathway and the one or more hydroperoxide reductase and catalase gene is TM1. The inducible promoter in the vector comprising the one or more genes of mevalonate pathway is TM3, the inducible promoter in another vector comprising the one or more genes of the mevalonate pathway is TM1, the inducible promoter in another vector comprising the one or more genes of the lycopene pathway is TM2 and the inducible promoter in another vector comprising the one or genes of the α-ionone pathway and the one or more hydroperoxide reductase and catalase gene is TM2. It is to be understood that the above examples are not exhaustive and are merely meant to illustrate that the polynucleotide sequences may be operably linked to different combinations of promoters.

In one example, the host cell is modified to not express at least one gene involved in amino acid synthesis. The host cell may be modified to not express one, two, three, four, five, six, seven, eight or more genes involved in amino acid synthesis. In another example, the host cell may be modified to express at least one gene involved in amino acid synthesis at a lower level compared to the baseline level. For example, the host cell may be modified to express one, two, three, four, five, six, seven, eight or more genes involved in amino acid synthesis at a lower level compared to the baseline level. The baseline level would be understood to mean the expression level of the at least one gene in an unmodified host cell. In one example, the at least one gene involved in amino acid synthesis includes but not limited to aroA, aroB, aroC and serC.

In one example, the host cell is a bacterial cell. In another example, the host cell is a bacterial cell that comprises a T7 polymerase. In yet another example, the bacterial cell is an Escherichia coli cell. The Escherichia coli cell may be a BL21 Gold DE3 strain, K-12 (RV308), K-12 (HMS174), K-12 substr. MG1655, W strain (ATCC 9637), JM109 (DE3), BW25113, JM109 DE3 or Mach1. In one example, the Escherichia coli cell is a BL21 Gold DE3 strain. In another example, the Escherichia coli cell is a BL21 Gold DE3 AaroABCAserC strain.

In one example, the Escherichia coli host cell comprises
a) a first vector comprising the polynucleotide sequence encoding atoB, hmgS and truncated hmgR genes of the mevalonate pathway and a CCD1 gene of the α-ionone pathway operably linked to a TM2 promoter;
b) a second vector comprising the polynucleotide sequence encoding mevk, pmk, pmd, idi genes of the mevalonate pathway operably linked to a TM3 promoter;
c) a third vector comprising the polynucleotide sequence encoding crtBI, IspA and the GGPPs gene of the lycopene pathway operably linked to a TM1 promoter; and
d) a fourth vector comprising the polynucleotide sequence encoding the LCYe, the CCD1 gene, of the α-ionone pathway and a hydroperoxide reductase gene operably linked to a TM1 promoter.

In another aspect, there is provided a method of producing one or more apocarotenoids comprising culturing the host cell as described herein in a culture medium.

In one example, α-ionone is further isolated from the culture medium.

The one or more apocarotenoids may include but not limited to α-ionone, β-ionone, psi-ionone, 6-methyl-5-heptene-2-one, hydroxy-ionone, retinol and retinal.

In one example, the method may produce at least one, at least two, at least three, at least four, at least five, at least six and at least seven apocarotenoids. In one preferred example, the at least one apocarotenoid is α-ionone.

In one example, the host cell is cultured in a culture medium in a snape tube or a batch culture medium in a flask or a bioreactor or a fed-batch fermentation culture medium. It would generally be understood that the host cell may be cultured under conditions suitable for growth and propagation of the host cell and production of apocarotenoids by the host cell. It would also generally be understood that the host cell may be cultured in culture medium that contains components suitable for growth and propagation of the host cell and production of apocarotenoids by the host cell.

In one example, the culture medium may comprise but not limited to an inducer and one or more carbon substrates. The culture medium may also comprise one or more antibiotics.

In one example, the inducer in the culture medium capable of inducing the inducible promoter linked to each of the vectors may be lactose or isopropyl β-D-1-thiogalactopyranoside (IPTG).

In one example, the one or more carbon substrate is glucose, glycerol or both. In one example, the at least one antibiotic may include but not limited to ampicillin, chloramphenicol, kanamycin and spectinomycin.

In one example, the culture medium in the snape tube or the batch culture medium in the flask or the bioreactor comprises glucose, glycerol, lactose, ampicillin, chloramphenicol, kanamycin and spectinomycin. In one example, the concentration of glucose is between about 1 g/L and about 10 g/L, the concentration of glycerol is between about 1 g/L and about 10 g/L, and the concentration of lactose is between about 10 mM and about 30 mM. The concentration of glucose may be about 1 g/L, about 2 g/L, about 3 g/L, about 4 g/L, about 5 g/L, about 6 g/L, about 7 g/L, about 8 g/L, about 9 g/L and about 10 g/L. In one example, the concentration of glycerol may be about 1 g/L, about 2 g/L, about 3 g/L, about 4 g/L, about 5 g/L, about 6 g/L, about 7 g/L, about 8 g/L, about 9 g/L and about 10 g/L. The concentration of lactose may be about 10 mM, about 15 mM, about 20 mM, about 25 mM and about 30 mM. In a preferred example, the concentration of glucose is about 2 g/L, the concentration of glycerol is about 8 g/L and the concentration of lactose is about 15 mM.

In one example, the host cell as described herein is cultured in a fed-batch fermentation culture medium comprising IPTG and glucose. In one example, the concentration of glucose is between about 1 g/L and about 10 g/L, and the concentration of IPTG is about 0.01 mM and about 0.2 mM. The concentration of glucose may be about 1 g/L, about 2 g/L, about 3 g/L, about 4 g/L, about 5 g/L, about 6 g/L, about 7 g/L, about 8 g/L, about 9 g/L and about 10 g/L. The concentration of IPTG may be about 0.01 mM, about 0.02 mM, about 0.03 mM, about 0.04 mM, about 0.05 mM, about 0.06 mM, about 0.07 mM, about 0.08 mM, about 0.09 nM, about 0.1 mM, about 0.11 mM, about 0.12 mM, about 0.13 mM, about 0.14 mM, about 0.15 mM, about 0.16 nM, about 0.17 nM, about 0.18 mM, about 0.19 mM and about 0.2 mM. In a preferred example, the concentration of glucose is about 5 g/L and the concentration of IPTG is about 0.1 mM.

The fed-batch fermentation culture medium may be supplemented with the carbon substrate during the growth of the host cell. For example, the fed-batch fermentation culture medium may be supplemented with carbon substrate, magnesium sulphate and inducer when the host cell has been cultured for a fixed duration or when the host cell has grown to an optimal density.

In one example, the fed-batch fermentation culture medium is supplemented with glucose at a concentration of between about 200 and about 700 g/L, and magnesium sulphate at a concentration of between about 1 g/L and about 10 g/L. The concentration of the glucose may be about 200 g/L, about 300 g/L, about 400 g/L, about 500 g/L, about 600 g/L and about 700 g/L. In one preferred example, the concentration of the glucose is about 500 g/L. In one example, the concentration of magnesium sulphate may be about 1 g/L, about 2 g/L, about 3 g/L, about 4 g/L, about 5 g/L, about 6 g/L, about 7 g/L, about 8 g/L, about 9 g/L and about 10 g/L. In a preferred example, the concentration of magnesium sulphate is about 5 g/L.

In one example, the fed-batch fermentation culture medium is further supplemented with the inducer when the host cell has grown to an optical density of about 10 to 50. The optical density may be about 10, about 15, about 20, about 25, about 30, about 35, about 40, about 45 and about 50. In one example, the optical density is about 40. In one example, the inducer is IPTG. The concentration of the IPTG is between about 0.01 mM and about 0.2 mM. The concentration of the IPTG is about 0.01 mM, about 0.02 mM, about 0.03 mM, about 0.04 mM, about 0.05 mM, about 0.06 mM, about 0.07 mM, about 0.08 mM, about 0.09 nM, about 0.1 mM, about 0.11 mM, about 0.12 mM, about 0.13 mM, about 0.14 mM, about 0.15 mM, about 0.16 nM, about 0.17 nM, about 0.18 mM, about 0.19 mM and about 0.2 mM. In a preferred example, the concentration of IPTG is about 0.1 mM.

In one example, the fed-batch fermentation culture medium is maintained at about pH 6.5 to about pH 7.5. In a preferred example, the fed-batch fermentation culture medium is maintained at about pH 7.

In one example, the host cell is cultured in the culture medium at a shaking speed of between about 50 rpm and about 2000 rpm. In one example, the host cell is cultured in the culture medium in the snape tube or the batch culture medium in the flask at a shaking speed of between about 50 rpm and about 500 rpm. In a preferred example, the host cell is cultured in the culture medium in the snape tube or the batch culture medium in the flask at a shaking speed of between about 100 and about 300 rpm. The host cell is cultured in the batch culture medium in the snape tube and flask at a shaking speed of about 300 rpm.

In another example, the host cell is cultured in the culture medium in a bioreactor at a shaking speed from between about 100 rpm and about 2000 rpm. In one example, the bioreactor is a personal bioreactor. It would be generally understood that personal bioreactors hold a small volume of culture media, for example, less than or about 50 mL, less than or about 40 mL, less than or about 30 mL, less than or about 20 mL, less than or about 10 mL, less than or about 5 mL, less than or about 2 mL or less than or about 1 mL. In a preferred example, the host cell is cultured in the culture medium in the bioreactor at a shaking speed from between about 250 rpm and about 500 rpm. In a preferred example, the host cell is cultured in the culture medium in the bioreactor at a shaking speed of about 500 rpm.

α-ionone may be extracted from the culture medium. In one example, the fed-batch fermentation culture medium is further supplemented with sunflower oil to extract α-ionone. In another example, the culture medium in the snape tube or the batch culture medium in the flask is supplemented with dodecane to extract α-ionone.

In one example, the yield of α-ionone in the fed-batch fermentation culture medium is at least 500 mg/L. The yield of α-ionone in the fed-batch fermentation culture medium is at least 500 mg/L at least 550 mg/L, at least 600 mg/L, at least 650 mg/L, at least 700 mg/L, at least 750 mg/L, at least 800 mg/L, at least 850 mg/L and at least 900 mg/L. In a preferred example, the yield of α-ionone is at least 700 mg/L.

In one example, the yield of α-ionone in the batch culture medium in the flask and the fed-batch fermentation culture medium is determined by titer per OD600 (titer/OD600). In one example, the titre per OD600 is between about 3 mg/L/OD₆₀₀-about 5 mg/L/OD₆₀₀. The titre per OD600 may be about 3 mg/L/OD₆₀₀, about 35 mg/L/OD₆₀₀, about 4 mg/L/OD₆₀₀, about 4.5 mg/L/OD₆₀₀ and about 5 mg/L/OD₆₀₀.

In another aspect, there is provided a kit for producing one or more apocarotenoids, wherein the kit comprises the host cell as described herein with instructions for use.

In one example, the host cell is dissolved in solution of lyophilized. In another example, the host cell is preserved by deep freezing.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Non-limiting examples of the invention and comparative examples will be further described in greater detail by reference to specific Examples, which should not be construed as in any way limiting the scope of the invention.

### Materials and Methods

### Bacteria Strains, Plasmids, and Oligonucleotides

The bacterial strain and plasmids used in this study are listed in Table 1. The specific oligonucleotides used for PCR amplification were synthesized by Integrated DNA Technologies (IDT) and listed in Table 2. Briefly, for construction of Module 4-7 (p15A-amp-ΔN50LsLCYe-OfCCD1-trxA (TM1) with three mutations of OfCCD1 active site loop, adding katG) and Module 4-8 (p15A-amp-ΔN50LsLCYe-OfCCD1-trxA (TM1) with three mutations of OfCCD1 active site loop, adding ahpC/F). The backbone was amplified with Module 4-1 as a template and the genes of the katG and ahpC/F was amplified from E. coli BL21. The cloning was performed using the iProof™ High-Fidelity DNA Polymerase (BIO-RAD).

TABLE 1
Strains and plasmids used in the study

| Strains | Description of plasmids |
|---|---|
| E. coli BI21 DE3 | E. coli BI21-Gold DE3 strain delete the gene aroA, aroB, aroC and |
| ΔaroABCΔserC | serC |
| AI_0000 | Module 1: p15A-spec-hmgS-atoB-hmgR (TM1) |
| | Module 2: p15A-cam-mevK-pmk-pmd-idi (TM2) |
| | Module 3: p15A-kan-crtEBI-ispA (TM1) |
| | Module 4: p15A-amp-ΔN50LsLCYe-OfCCD1-trxA(TM1) |
| AI_2211 | Module 1: p15A-spec-hmgS-atoB-hmgR- OfCCD1-trxA (TM2) |
| | Module 2: p15A-cam-mevK-pmk-pmd-idi (TM3) |
| | Module 3: p15A-kan-crtBI-ispA-MbGGPPs (TM1) |
| | Module 4: p15A-amp-ΔN50LsLCYe-OfCCD1-trxA(TM1) with |
| | three mutations of OfCCD1 active site loop |
| AI_3211 | Module 1: p15A-spec-hmgS-atoB-hmgR (TM2) |
| | Module 2: p15A-cam-mevK-pmk-pmd-idi (TM3) |
| | Module 3: p15A-kan-crtBI-ispA-MbGGPPs (TM1) |
| | Module 4: p15A-amp-ΔN50LsLCYe-OfCCD1-trxA(TM1) with |
| | three mutations of OfCCD1 active site loop |
| AI_2217 | Module 1: p15A-spec-hmgS-atoB-hmgR- OfCCD1-trxA (TM2) |
| | Module 2: p15A-cam-mevK-pmk-pmd-idi (TM3) |
| | Module 3: p15A-kan-crtBI-ispA-MbGGPPs (TM1) |
| | Module 4: p15A-amp-ΔN50LsLCYe-OfCCD1-trxA(TM1) with |
| | three mutations of OfCCD1 active site loop, adding katG. |
| AI_2218 | Module 1: p15A-spec-hmgS-atoB-hmgR- OfCCD1-trxA (TM2) |
| | Module 2: p15A-cam-mevK-pmk-pmd-idi (TM3) |
| | Module 3: p15A-kan-crtBI-ispA-MbGGPPs (TM1) |
| | Module 4: p15A-amp-ΔN50LsLCYe-OfCCD1-trxA(TM1) with |
| | three mutations of OfCCD1 active site loop, adding ahpC/F. |

TABLE 2
Oligonucleotides used for PCR amplifications

| | Oligo- | |
|---|---|---|
| | nucleotide | |
| Primer name | sequence | Function |
| P2_Catalase_F | gtttgctgcc | Amplified as |
| (SEQ ID NO: 6) | accgctgagc | a backbone |
| | | from Module 4-1 |
| P2_Catalase_R | ttacactttg | |
| (SEQ ID NO: 7) | gcctgttcct | |
| AhpC_F_IV | aggaacaggc | Amplified ahpC/F |
| (SEQ ID NO: 8) | caaagtgtaa | from E.coli |
| | gtaaggtaaa | BL21 |
| | acttatcgat | |
| AhpF_R_IV | gctcagcggt | |
| (SEQ ID NO: 9) | ggcagcaaac | |
| | ttatgcagtt | |
| | ttggtgcgaa | |
| KatG_F_IV | aggaacaggc | Amplified katG |
| | caaagtgtaa | from E.coli |
| (SEQ ID NO: 10) | ccaacaatat | BL21 |
| | gtaagatctc | |
| KatG_R_IV | gctcagcggt | |
| | ggcagcaaac | |
| (SEQ ID NO: 11) | ttacagcagg | |
| | tcgaaacggt | |
| Catalase_Check_F | ccggttgcag | To determine if |
| (SEQ ID NO: 12) | tagtcgaact | Modules 4-7 |
| | gc | and 4-8 are |
| Catalase_Check_R | gccggtactg | successfully |
| (SEQ ID NO: 13) | ccgggcctctt | constructed |

### Media and Culture Conditions

All the cells were grown in LB media. For the production test, a chemically defined Auto-induction medium was used, which contained 2 g/L glucose and 8 g/L glycerol, 2 g/L ammonium sulfate, 4.2 g/L KH₂PO₄, 11.24 g/L K₂HPO₄, 1.7 g/L citric acid, 0.5 g/L MgSO₄, and 10 mL/L trace element solution. The trace element solution (100×) contained 0.25 g/L CoCl₂·6H₂O, 1.5 g/L MnSO₄·4H₂O, 0.15 g/L CuSO₄·2H₂O, 0.3 g/L H₃BO₃, 0.25 g/L Na₂MoO₄·2H₂O, 0.8 g/L Zn(CH₃COO)₂, 5 g/L Fe(III) citrate, and 0.84 g/L ethylenediaminetetraacetic acid (EDTA) at pH 8.0. Cells were induced by 15 mM Lactose. Briefly, 1% fresh cell culture was inoculated into 1 ml defined Auto-induction medium in 14 ml snape cap tubes falcon or 10 ml defined Auto-induction medium in 100 ml Flask. After induction, 200 μl for 1 ml culture and 10 ml for 10 ml culture of dodecane was supplemented onto the culture to extract ionone, and the cells were incubated at 28° C. for 72 hr with the shaking speed of 100 rpm or 300 rpm before harvest. The media was supplemented with appropriate antibiotics (100 mg/L ampicillin, 34 mg/L chloramphenicol, 50 mg/L kanamycin, and 50 mg/L spectinomycin) to maintain corresponding plasmids.

### Quantification of α-Ionone and MHO

The α-ionone, or MHO samples were prepared by diluting 10-50 times of organic layer into 1,000 μl hexane. GC-MS analysis of the samples was performed on an Intuvo 9000 GC system attached with a 5977B MS detector (Agilent Technologies, USA). The system was equipped with a polar DB wax column (polyethylene glycol (PEG); 30 m×0.25 mm I.D.×0.25 μm: Agilent Technologies, USA) and a split injector (split ratio 1:10). The oven program started at 80° C. for 1 min, then the temperature was raised up at 20° C./min until 130° C. hold time 1.5 min and raised up at 40° C./min until 200° C. hold time 2 min, final raised up at 80° C./min maintained at 230° C. for another 2 min. Helium gas was used as carrier gas at a constant flow rate of 1.0 mL/min. The Agilent 5977B mass spectrometer was operated in the electron ionization mode at 70 eV with a source temperature of 230° C., transfer line temperature set at 250° C., and a scan range of m/z 50-500 in the full scan mode at an acquisition rate of 3.6 scans/s. The solvent for column wash was methanol while hexane was used for needle wash. The injection volume was 1 μL. The ionone concentrations were calculated by interpolating with a standard curve prepared by commercial standards. Mass spectrometer was operated in EI mode with full scan analysis.

ROS-Glo™ H₂O₂ Assay

ROS-Glo (Promega, Madison, WI, USA) assay was used as specified by the vendor to quantify H₂O₂ production. Inoculated AI_2211 and AI_2218 in 1 ml auto-induced R-medium by RTS-1C (Personal Bioreactor, Biosan). Cells were grown to an OD₆₀₀ of 1.5-2 and then the H₂O₂ substrate was added. Subsequently, the culture was incubated for 1.5 hr and the cells were then harvested and the amount of luciferin precursor produced measured. The H₂O₂ substrate reacts directly with H₂O₂ to generate a luciferin precursor. Upon addition of ROS-Glo™ Detection Reagent containing Ultra-Glo™ Recombinant Luciferase and d-Cysteine, the precursor is converted to luciferin by the d-Cysteine, and the produced luciferin reacts with Ultra-Glo™ Recombinant Luciferase to generate a luminescent signal that is proportional to H₂O₂ concentration. (ROS-Glo™ H₂O₂ Assay, Promega). Briefly, 800 μl of cells was incubated with 200 μl of H₂O₂ substrate followed by addition of the ROS-Glo detection reagent. Luminescence corresponding to H₂O₂ levels was measured using micro plate reader (Molecular Devices, San Jose, CA, USA).

### Fed-Batch Fermentation

Starting medium was a modified chemically defined medium, which contained 5 g/L of glucose and was transferred into a 5-L bioreactor with the initial working volume of 1.8 L. The chemical defined medium also contained 2 g/L (NH₄)₂SO₄, 4.2 g/L KH₂PO₄, 11.24 g/L K₂HPO₄, 1.7 g/L citric acid, 0.5 g/L MgSO₄ and 10 ml/l trace element solution, pH 7.0. The trace element solution (100×) contained 0.25 g/L CoCl₂·6H₂O, 1.5 g/L MnSO₄·4H₂O, 0.15 g/L CuSO₄· 2H₂O, 0.3 g/L H₃BO₃, 0.25 g/L Na₂MoO₄·2H₂O, 0.8 g/L Zn(CH₃COO)₂, 5 g/L Fe(III) citrate and 0.84 g/L EDTA, pH 8.0. The E. coli strain AI_2218 was inoculated into the sterile defined medium to obtain the initial optical density at 600 nm (or OD600) of 0.1. The fermentation was first carried out under the controlled set point of pH, temperature and dissolved oxygen at 7.0, 37° C. and 30%, respectively. After inoculation, peristatic pump of the feed stock solution (containing 500 g/L glucose and 5 g/L MgSO4) was also started at 1.62 mL/h of flow rate for overnight (˜13 h). The pH of the culture was controlled at 7.0 using alkaline solution (the mixture of 28% ammonium hydroxide and 1M sodium hydroxide solution; in ratio 1:1 by volume) throughout the experiments. Cells were induced by 0.1 mM IPTG when OD600 reached about 40 and the 500 mL of sunflower oil as extractant was then added into the bioreactor.

### Results

### Example 1

### Oxygen Sensitivity of New Strain: AI_2211

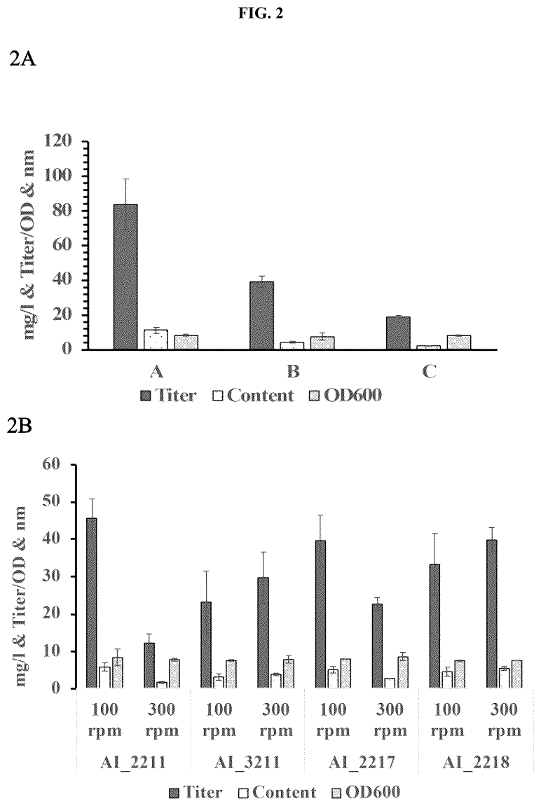

Previously, biosynthetic pathway was constructed for α-ionone production in E. coli. This biosynthetic pathway consists of 4 modules includes: 1. upstream mevalonate pathway, 2. downstream mevalonate pathway 3. lycopene pathway and 4. α-ionone pathway. Based on how this pathway is modified, the α-ionone producing strains were named as AI_ABCD (Table 1). To improve the production of α-ionone, an additional copy of CCD1 was overexpressed to enhance the ε-carotene conversion to α-ionone and the expression levels of pathway genes were re-optimized. The AI_2211 strain was tested under three different conditions, A: 1 ml defined medium, 300 rpm in Snape tube. B: 10 ml defined medium, 100 rpm by flask. C: 10 ml defined medium, 300 rpm by flask, to understand the yield of the strain when we scaled up from 1 ml to 10 ml (FIG. 2A). The results indicated that, as the shaking speed was increased, both titers and contents of α-ionone were decreased. It is unclear if the observed negative effect of shaking speeds on α-ionone production was due to an additional copy of CCD1 on the plasmids present in AI_2211. Thus, to test this hypothesis, the extra CCD1 was removed while maintaining the rest of genes the same as AI_2211, to obtain the AI_3211 strain (Table 1). These two strains were compared under 100 rpm and 300 rpm in flasks. Interestingly, AI_3211 strain produced higher amount of α-ionone at 300 rpm than 100 rpm. In contrast, AI_2211 strain produced around 4-fold higher amount of α-ionone at 100 rpm than 300 rpm (FIG. 2B). Higher shaking speeds contributed to higher amount of dissolved oxygen and potentially higher oxidative stress in flasks. Hence, it was hypothesized that this phenomenon might be attributed to the generation of reactive oxygen species (ROSs), especially H₂O₂.

H₂O₂ Production of In Vivo Overexpression of Catalase Genes; Catalase G (katG): AI_2217 and Alkyl Hydroperoxide Reductase (ahpC/F): AI_2218

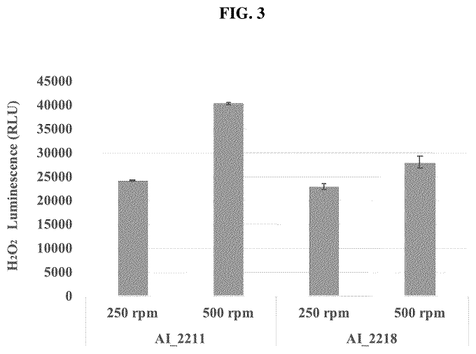

To test the hypothesis, katG (catalase G) was overexpressed as AI_2217, and ahpC/F (alkyl hydroperoxide reductase) was overexpressed as AI_2218 (Table 1). Based on the comparative results of these two strains, AI_2218 gave higher α-ionone titers when the shaking speed was high. However, AI_2217 shows the same trend as AI_2211 but with around 2-fold higher amount of α-ionone at 100 rpm than 300 rpm (FIG. 2B). In summary, α-ionone titers of different strains (AI_2211, AI_3211, AI_2217 and AI_2218) were compared. The results showed that α-ionone titers of AI_3211 and AI_2218 were positively correlated with the shaking speed, and in contrast, α-ionone titers of AI_2211 and AI_2217 were negatively correlated (FIG. 2B). Furthermore, the H₂O₂ production was measured by ROS-Glo™ H₂O₂ Assay with AI_2211 and AI_2218 in 1 ml defined medium. Due to the small amount of the medium, instead of using flask, the experiment was performed under 250 and 500 rpm by RTS-1C. The results showed, H₂O₂ concentration was around 40000 RLU under AI_2211 with 500 rpm, which is 1.6 fold higher than AI_2211 with 250 rpm and AI_2218 with both shaking speeds. (FIG. 3). The measurement of H₂O₂ concentration demonstrated that at high agitation rate, AI_2211 is associated with more oxidative stress by H₂O₂; moreover, it also proved that AI_2218 eliminates H₂O₂ more efficiently because of ahpC/F overexpression.

### Apocarotenoid: MHO Production by AI_2211 and AI_2218 Under Different Shaking Speed Conditions

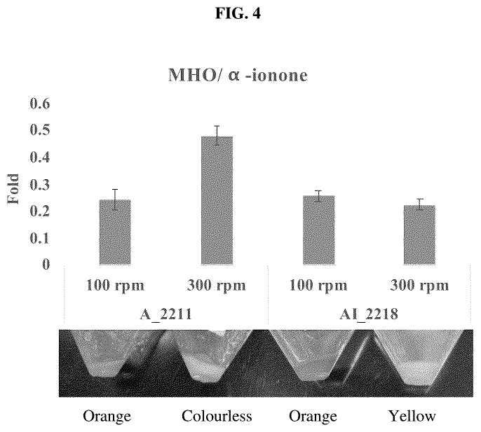

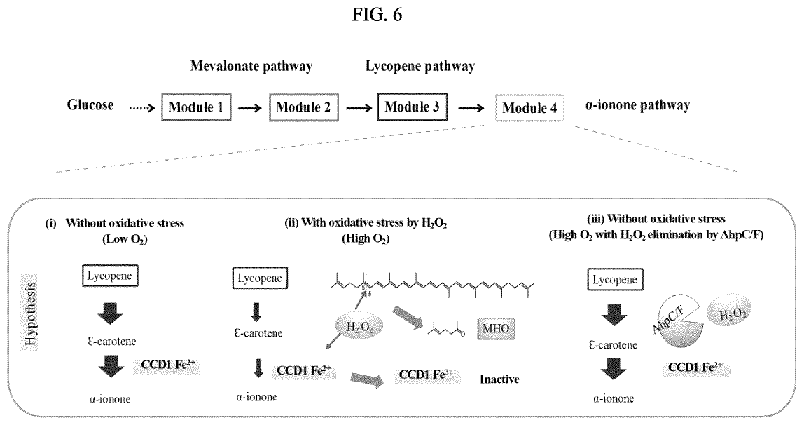

Next, the effect of H₂O₂ on the pathway and the further decrease of the production of α-ionone in AI_2211 were investigated. From previous experiments, it was observed that the colors of the pellets of AI_2211 were orange and white under 100 rpm and 300 rpm, respectively (FIG. 4). Naturally occurring Lycopene is red and ε-carotene is yellow in color. Thus, it is assumed the H₂O₂ might degrade some of the intermediate compounds of the pathway, such as lycopene and ε-carotene, which would reduce the pathway flux towards α-ionone production. From the experiment with AI_2211 and AI_2218 inoculated under 100 rpm and 300 rpm, it was observed there is 6-Methyl-5-hepten-2-one (MHO), which is an important flavor and aroma volatile found in a number of fruits such as tomato. The production of MHO with α-ionone was compared and the cell pellet was collected to analyze the different colors. The MHO to α-ionone ratio of the AI_2211 under 300 rpm is around 0.5 which is two times higher than all the other tests done (around 0.2-0.3) (FIG. 4). These results indicated that for the AI_2211 strain, the concentration of MHO does increase under higher shaking speed rate and is also associated to a decrease of α-ionone production. Moreover, the substrate degradation can be proven from the pellet color of AI_2211 under 300 rpm which is colorless compared to AI_2211 at 100 rpm and AI_2218 at 300 rpm. Orange color of the pellet of AI_2211 and AI_2218 under 100 rpm showed that the pellet may mix with lycopene and ε-carotene. The pellet color of AI_2218 at 300 rpm which is yellow showed that most of them are ε-carotene (FIGS. 4 and 6).

### AI_2218, Bioreactor Fermentation for α-Ionone Production

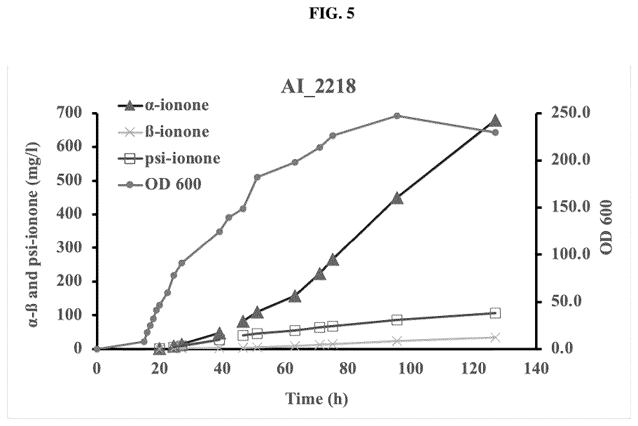

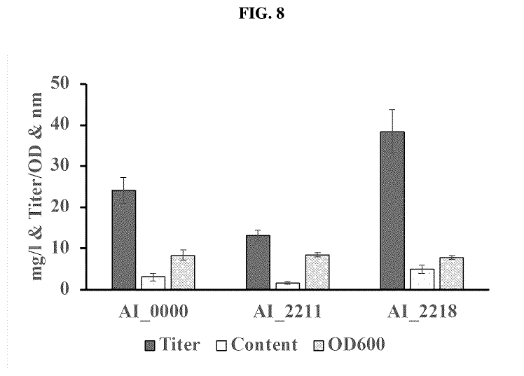

Before scaling up to 5 L bioreactor, the strains in flask: AI_0000, AI_2211 and AI_2218 under 300 rpm were compared to confirm which strain can produce higher α-ionone titers (FIG. 8). The results showed AI_2218 obtained the highest α-ionone titers as compared to AI_0000 and AI_2211. In contrast, AI_2211 produced the highest amount of α-ionone at 100 rpm. However, due to its extremely high sensitivity towards oxygen, scaling up the AI_2211 strain may be challenging. In comparison, AI_2218 produced the highest amount of α-ionone at high shaking speed rate, which was the most promising condition for the scale-up. Subsequently, the 5 L bioreactor experiment has been undertaken with AI_2218 and was shown to produce approximately 700 mg/l α-ionone (FIG. 5).

The polypeptide and polynucleotide sequences used in the present invention is summarized in the table below.

TABLE 3
Summary of sequence listing

| | Polypeptide Sequence or |
|---|---|
| Description | Polynucleotide Sequence |
| Polypeptide | MGMQGEDAQRTGNIVAVKPKPSQGLTSKAI |
| sequence | DWLEWLFVKMMHDSKQPLHYLSGNFAPVDE |
| of OfCCD1 | TPPLKDLPVTGHLPECLNGEFVRVGPNPKF |
| (SEQ ID NO: 1) | ASIAGYHWFDGDGMIHGMRIKDGKATYVSR |
| | YVQTSRLKQEEFFGRAMFMKIGDLKGMFGL |
| | LMVNMQMLRAKLKVLDISYGIGTANTALVY |
| | HHGKLLALSEADKPYAIKVLEDGDLQTIGL |
| | LDYDKRLAHSFTAHPKVDPFTGEMFTFGYS |
| | HTPPYVTYRVISKDGAMNDPVPITVSGPIM |
| | MHDFAITENYAIFMDLPLYFKPKEMVKDKK |
| | FIFSFDATQKARFGILPRYAKNELLIKWFE |
| | LPNCFIFHNANAWEEGDEVVLITCRLENPD |
| | LDMVNSTVKERLDNFKNELYEMRFNLONGL |
| | ASQKKLSVSAVDFPRVNESYTTRKQRYVYG |
| | TTLDKIAKVTGIIKFDLHAEPETGKEKLEL |
| | GGNVKGIFDLGPGRFGSEAVFVPRHPGITS |
| | EEDDGYLIFFVHDENTGKSAVNVIDAKTMS |
| | PDPVAVVELPKRVPYGFHAFFVTEDQLQEQ |
| | AKV |
| Polynucleotide | atgagcgataaaattattcacctgactgac |
| sequence | gacagttttgacacggatgtactcaaagcg |
| of mutated | gacggggcgatcctcgtcgatttctgggca |
| OfCCD1 (SEQ ID | gagtggtgcggtccgtgcaaaatgatcgcc |
| NO: 2) | ccgattctggatgaaatcgctgacgaatat |
| | cagggcaaactgaccgttgcaaaactgaac |
| | atcgatcaaaaccctggcactgcgccgaaa |
| | tatggcatccgtggtatcccgactctgctg |
| | ctgttcaaaaacggtgaagtggcggcaacc |
| | aaagtgggtgcactgtctaaaggtcagttg |
| | aaagagttcctcgacgctaacctggcgggt |
| | cgtaaagaaagcgacgacggcgttgaacgc |
| | atcgaaggtggcgtggttgttgtaaaccca |
| | aaaccaaagaaaggcatcactgcatcgaaa |
| | gcaattgactggctggagtggctgtttgtt |
| | aaaatgatgcatgacagcaaacagccgctg |
| | cactacttgtccggtaacttcgcaccggtt |
| | gacgaaaccccgccgctgaaagacctgccg |
| | gtgaccggccatctgccggaatgcctgaac |
| | ggcgagtttgtgcgtgtcggtccgaacccg |
| | aaattcgcttccatcgccggttaccactgg |
| | tttgacggtgatggtatgatccatggtatg |
| | cgcattaaggacggtaaagccacctatgtt |
| | tctcgttatgttcagacctcccgcctcaaa |
| | caggaagaattcttcggtcgtgctatgttt |
| | atgaaaattggagatctgaaaggtatgttc |
| | ggcctgttcaccgtttacatgcagatgctg |
| | cgtgcgaaactgaaagtcctggacgcatct |
| | tacggtattggtaccgccaacaccgcgctg |
| | gtttaccaccatggcaaactgctcgctctg |
| | agcgaagctgacaaaccgtatgcgatcaaa |
| | gttctggaagatggcgacctgcagaccatc |
| | ggcctgttagattacgacaaacgcctggca |
| | catagtttcaccgcacatcccaaggttgac |
| | ccgtttacaggcgaaatgttcacgttcggt |
| | tattcccacaccccgccgtatgtgacctac |
| | cgtgttatctctaaagatggtgcaatgaac |
| | gatccggtacctatcacggtctccggccca |
| | atcatgatgcacgacttcgccatcacggaa |
| | aactacgctattttcatggatttgccgctg |
| | tacttcaaaccaaaagagatggtgaaagat |
| | aaaaagttcatcttcagcttcgacgcaacc |
| | cagaaggctcgctttggcatcctgccgcgc |
| | tacgccaaaaacgaatctctgatcaaatgg |
| | ttcgagctcccgaactgcttcatctttcac |
| | aatgcgaacgcttgggaagagggtgacgaa |
| | gttgtccttattacctgccgtctggaaaac |
| | ccggacctggacatggttaacagcaccgtt |
| | aaagaacgtctggacaacttcaaaaacgaa |
| | ctgtacgaaatgcgcttcaacctgcagaac |
| | ggtctggctagccagaaaaaactgagcgta |
| | agcgcggttgattttccgcgtgttaacgaa |
| | tcgtacaccacccgtaaacagcgttatgtt |
| | tatggtacgaccctggataacatcgcgaaa |
| | gttaccggtatcatcaaattcgacctgcac |
| | gccgaaccggaaacgggcaaagaaaagctg |
| | gaactgggtggcaacgtaaaaggtattttt |
| | gatctgggcccgggtcgcttcggtagtgaa |
| | gccgttttcgttccacgtcacccgggcatc |
| | acctccgaagaagatgacggttatctcatt |
| | tttttcgtgcatgatgagaacacaggtaaa |
| | tctgcagtaaacgtgatcgacgcaaaaacc |
| | atgtctcctgacccggttgcagtagtcgaa |
| | ctgccgaaacgcgtgccatacggctttcac |
| | gcttttttcgtaaccgaagaccagctgcag |
| | gaacaggccaaagtgtaa |
| Polynucleotide | atgaagtgctccgcaaaatccgaccgttgc |
| sequence of | gttgttgacaaacaagggattagcgtggct |
| truncated LCYe | gatgaggaagactacgttaaggcaggcggt |
| (SEQ ID NO: 3) | tccgaacttttctttgtgcagatgcagcgc |
| | accaagtccatggaatcccagtccaaactg |
| | tccgaaaaactggcgcagattccgattggc |
| | aattgcatccttgacctggtggttattggt |
| | tgtggcccggcaggtctggcgcttgcggct |
| | gagtccgcaaaactgggtctgaacgttggc |
| | ctcatcggtccggacctcccattcaccaac |
| | aactacggcgtttggcaggacgaattcatc |
| | ggtctcggtctggaaggctgtatcgaacac |
| | tcctggaaagacaccctggtgtacctggat |
| | gatgcggacccaatccgtatcggccgtgcg |
| | tacggccgcgtgcaccgtgacctgctgcac |
| | gaagaactcctgcgtcgctgtgtggagtct |
| | ggtgttagctatctgagttccaaggtagag |
| | cgtattaccgaagctccaaacggctacagc |
| | ctgatcgaatgcgaaggtaacatcactatc |
| | ccgtgtcgtctggcaaccgtggcgtccggc |
| | gcggcgagcggtaaattcctggaatacgaa |
| | ctggggggccgcgtgtttgcgttcagaccg |
| | cttacggtattgaagtagaagttgaaaaca |
| | acccgtacgatccggatctgatggtattta |
| | tggattaccgtgacttctcaaaacataaac |
| | cggaaagcctggaagctaaataccctacct |
| | tcctgtatgttatggcgatgtctccgacca |
| | aaatcttcttcgaggagacctgcctggcgt |
| | cccgcgaagcaatgccgttcaacctgctga |
| | aaagcaaactgatgagccgtctgaaagcca |
| | tgggtatccgtattacccgtacctatgaag |
| | aagaatggagctatatcccggtaggtggct |
| | ctctcccgaacacggaacagaaaaacctgg |
| | cgttcggcgcggctgcatccatggttcacc |
| | cggctactggctactccgttgttcgttccc |
| | tgtctgaagccccgaactatgcagctgtaa |
| | tcgctaaaatcctgcgtcaggatcagtcta |
| | aggaaatgatttctctcggtaaatacacca |
| | acatttccaaacaggcgtgggaaaccctct |
| | ggccgctggaacgcaaacgccagcgtgcat |
| | ttttcctcttcggtctgtcccacatcgtgc |
| | tcatggatctggaaggcacccgtaccttct |
| | tccgcaccttctttcgcctgccgaaatgga |
| | tgtggtggggcttcctcggttcctctctgt |
| | cttctaccgacctgatcatcttcgctctgt |
| | acatgttcgtaatcgcgccgcacagcctgc |
| | gcatggaactggttcgtcacctgctgtcgg |
| | acccgaccggtgctaccatggtaaaagcat |
| | acttaactatt |
| Polynucleotide | ttacactttggcctgttcctgcagctggtc |
| sequence | ttcggttacgaaaaaagcgtgaaagccgta |
| of wild- | tggcacgcgtttcggcagttcgactactgc |
| type OfCCD1 | aaccgggtcaggagacatggtttttgcgtc |
| (SEQ | gatcacgtttactgcagatttacctgtgtt |
| ID NO:4) | ctcatcatgcacgaaaaaaatgagataacc |
| | gtcatcttcttcggaggtgatgcccgggtg |
| | acgtggaacgaaaacggcttcactaccgaa |
| | gcgacccgggcccagatcaaaaataccttt |
| | tacgttgccacccagttccagcttttcttt |
| | gcccgtttccggttcggcgtgcaggtcgaa |
| | tttgatgataccggtaactttcgcgatttt |
| | atccagggtcgtaccataaacataacgctg |
| | tttacgggtggtgtacgattcgttaacacg |
| | cggaaaatcaaccgcgcttacgctcagttt |
| | tttctggctagccagaccgttctgcaggtt |
| | gaagcgcatttcgtacagttcgtttttgaa |
| | gttgtccagacgttctttaacggtgctgtt |
| | aaccatgtccaggtccgggttttccagacg |
| | gcaggtaataaggacaacttcgtcaccctc |
| | ttcccaagcgttcgcattgtgaaagatgaa |
| | gcagttcgggagctcgaaccatttgatcag |
| | cagttcgtttttggcgtagcgcggcaggat |
| | gccaaagcgagccttctgggttgcgtcgaa |
| | gctgaagatgaactttttatctttcaccat |
| | ctcttttggtttgaagtacagcggcaaatc |
| | catgaaaatagcgtagttttccgtgatggc |
| | gaagtcgtgcatcatgattgggccggagac |
| | cgtgataggtaccggatcgttcattgcacc |
| | atctttagagataacacggtaggtcacata |
| | cggcggggtgtgggaataaccgaacgtgaa |
| | catttcgcctgtaaacgggtcaaccttggg |
| | atgtgcggtgaaactatgtgccaggcgttt |
| | gtcgtaatctaacaggccgatggtctgcag |
| | gtcgccatcttccagaactttgatcgcata |
| | cggtttgtcagcttcgctcagagcgagcag |
| | tttgccatggtggtaaaccagcgcggtgtt |
| | ggcggtaccaataccgtaagagatgtccag |
| | gactttcagtttcgcacgcagcatctgcat |
| | gttaaccatcagcaggccgaacataccttt |
| | cagatctccaattttcataaacatagcacg |
| | accgaagaattcttcctgtttgaggcggga |
| | ggtctgaacataacgagaaacataggtggc |
| | tttaccgtccttaatgcgcataccatggat |
| | cataccatcaccgtcaaaccagtggtaacc |
| | ggcgatggaagcgaatttcgggttcggacc |
| | gacacgcacaaactcgccgttcaggcattc |
| | cggcagatggccggtcaccggcaggtcttt |
| | cagcggcggggtttcgtcaaccggtgcgaa |
| | gttaccggacaagtagtgcagcggctgttt |
| | gctgtcatgcatcattttaacaaacagcca |
| | ctccagccagtcaattgctttcgaagtcag |
| | tccctgggaaggtttcggtttcacggcaac |
| | aatgttacctgtacgctgtgcgtcttcacc |
| | ctgcattcccgccaggttagcgtcgaggaa |
| | ctctttcaactgacctttagacagtgcacc |
| | cactttggttgccgccacttcaccgttttt |
| | gaacagcagcagagtcgggataccacggat |
| | gccatatttcggcgcagtgccagggttttg |
| | atcgatgttcagttttgcaacggtcagttt |
| | gccctgatattcgtcagcgatttcatccag |
| | aatcggggcgatcattttgcacggaccgca |
| | ccactctgcccagaaatcgacgaggatcgc |
| | cccgtccgctttgagtacatccgtgtcaaa |
| | actgtcgtcagtcaggtgaataattttatc |
| | gctcat |
| Polynucleotide | gaaacaaatgacgtgaaagttcttcaaaat |
| sequence | tgaattaattgtaatcctgaaaacttgatt |
| of wild- | tgtgatagaagaatcaatggagtgctttgg |
| type LCYe | agctcgaaacatgacggcaacaatggcggt |
| gene | ttttacgtgccctagattcacggactgtaa |
| (SEQ ID NO: 5) | tatcaggcacaaattttcgttactgaaaca |
| | acgaagatttactaatttatcagcatcgtc |
| | ttcgttgcgtcaaattaagtgcagcgctaa |
| | aagcgaccgttgtgtagtggataaacaagg |
| | gatttccgtagcagacgaagaagattatgt |
| | gaaggccggtggatcggagctgttttttgt |
| | tcaaatgcagcggactaagtccatggaaag |
| | ccagtctaaactttccgaaaagctagcaca |
| | gataccaattggaaattgcatacttgatct |
| | ggttgtaatcggttgtggccctgctggcct |
| | tgctcttgctgcagagtcagccaaactagg |
| | gttgaacgttggactcattggccctgatct |
| | tccttttacaaacaattatggtgtttggca |
| | ggatgaatttataggtcttggacttgaagg |
| | atgcattgaacattcttggaaagatactct |
| | tgtataccttgatgatgctgatcccatccg |
| | cataggtcgtgcatatggcagagttcatcg |
| | tgatttacttcatgaagagttgttaagaag |
| | gtgtgtggaatcaggtgtttcatatctaag |
| | ctccaaagtagaaagaatcactgaagctcc |
| | aaatggctatagtctcattgaatgtgaagg |
| | caatatcaccattccatgcaggcttgctac |
| | tgttgcatcaggggcagcttcagggaaatt |
| | tctggagtatgaacttgggggtccccgtgt |
| | ttgtgtccaaacagcttatggtatagaggt |
| | tgaggttgaaaacaacccctatgatccaga |
| | tctaatggtgttcatggattatagagactt |
| | ctcaaaacataaaccggaatctttagaagc |
| | aaaatatccgactttcctctatgtcatggc |
| | catgtctccaacaaaaatattcttcgagga |
| | aacttgtttagcttcaagagaagccatgcc |
| | tttcaatcttctaaagtccaaactcatgtc |
| | acgattaaaggcaatgggtatccgaataac |
| | aagaacgtacgaagaggaatggtcgtatat |
| | ccccgtaggtggatcgttacctaatacaga |
| | acaaaagaatctcgcatttggtgctgcagc |
| | tagtatggtgcaccctgccacagggtattc |
| | agttgttcgatctttgtcagaagctcctaa |
| | ttatgcagcagtcattgctaagattttaag |
| | acaagatcaatctaaagagatgatttctct |
| | tggaaaatacactaacatttcaaaacaagc |
| | atgggaaacattgtggccacttgaaaggaa |
| | aagacagcgagccttctttctattcggact |
| | atcacacatcgtgctaatggatctagaggg |
| | aacacgtacatttttccgtactttctttcg |
| | tttgcccaaatggatgtggtggggattttt |
| | ggggtcttctttatcttcaacggatttgat |
| | aatatttgcgctttatatgtttgtgatagc |
| | acctcacagcttgagaatggaactggttag |
| | acatctactttctgatccgacaggggcaac |
| | tatggtaaaagcatatctcactatatagat |
| | ttagattatataaataatacccatatcttg |
| | catatatataagccttatttatttcttttg |
| | tatccttacaacaacatactcgttaattat |
| | atgtttttta |
| Polynucleotide | gtttgctgccaccgctgagc |
| sequence | |
| of Forward | |
| Primer for | |
| Catalase | |
| (SEQ ID NO: 6) | |
| Polynucleotide | ttacactttggcctgttcct |
| sequence | |
| of Reverse | |
| Primer for | |
| Catalase | |
| (SEQ ID NO: 7) | |
| Polynucleotide | aggaacaggccaaagtgtaa gtaaggtaa |
| sequence of | aacttatcgat |
| Forward | |
| Primer for | |
| AhpC | |
| (SEQ ID NO: 8) | |
| Polynucleotide | gctcagcggtggcagcaaac ttatgcagt |
| sequence of | tttggtgcgaa |
| Reverse | |
| Primer for | |
| AhpC | |
| (SEQ ID NO: 9) | |
| Polynucleotide | aggaacaggccaaagtgtaa ccaacaata |
| sequence of | tgtaagatctc |
| Forward | |
| Primer for | |
| KatG | |
| (SEQ ID NO: 10) | |
| Polynucleotide | gctcagcggtggcagcaaac ttacagcag |
| sequence of | gtcgaaacggt |
| Reverse | |
| Primer for Kat G | |
| (SEQ ID NO: 11) | |
| Polynucleotide | ccggttgcagtagtcgaactgc |
| sequence of | |
| Forward | |
| Primer for | |
| Catalase | |
| Check (SEQ ID NO: | |
| 12) | |
| Polynucleotide | gccggtactgccgggcctctt |
| sequence of | |
| Reverse | |
| Primer for | |
| Catalase | |
| Check (SEQ ID NO: | |
| 13) | |

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1.-37. (canceled)

1. A host cell comprising one or more vectors comprising a polynucleotide sequence encoding:
one or more genes of the mevalonate pathway;
one or more genes of the lycopene pathway;
one or more genes of the α-ionone pathway; and
one or more hydroperoxide reductase or catalase genes.

2. The host cell according to claim 1, wherein the one or more genes of the mevalonate pathway are selected from the group consisting of hmgS, atoB, hmgR, mevK, pmk, pmd and idi, optionally wherein the hmgR gene is truncated.

3.-37. (canceled)

38. The host cell according to claim 1, wherein the one or more genes of the lycopene pathway are selected from the group consisting of crtBI, crtB, crtI, a farnesyl diphosphate synthase gene and a geranylgeranyl diphosphate synthase (GGPPs) gene, optionally wherein the GGPPs gene is isolated from a prokaryote, wherein the prokaryote is archaea comprising Methanocaldococcus jannaschii or Methanococcus maripaludis, or bacteria comprising Methanobacterium or Deinococcus radiodurans, optionally wherein the bacterium is a methanobacterium.

39. The host cell according to claim 1, wherein the one or more genes of the α-ionone pathway are selected from the group consisting of a lycopene cyclase gene and a CCD gene.

40. The host cell according to claim 1, wherein the one or more vectors further comprises a polynucleotide sequence encoding one or more additional carotenoid cleavage dioxygenase (CCD) genes of the α-ionone pathway.

41. The host cell according to claim 39, wherein the CCD gene is a CCD1 or CCD4 gene, or a combination thereof, optionally wherein one or more of the CCD1 genes is isolated from a plant selected from the group consisting of Osmanthus fragrans, Arabidopsis thaliana, Vitis vinifera and Petunia hybrid or combinations thereof, optionally wherein the one or more of the CCD1 genes is isolated from Osmanthus fragrans, optionally wherein the one or more of the CCD1 genes isolated from Osmanthus fragrans (OfCCD1) is fused with an N-terminal fusion partner, optionally wherein the one or more of the OfCCD1 genes comprises the polypeptide sequence set forth in SEQ ID NO: 1.

42. The host cell according to claim 41, wherein the one or more of the OfCCD1 genes is mutated at the loop of the active site, optionally wherein the mutation is at one or more amino acid positions, optionally wherein the one or more of the mutated OfCCD1 genes comprises the polynucleotide sequence set forth in SEQ ID NO: 2; optionally wherein the mutation is a substitution of lysine at position 425 with serine, a substitution of aspartate at position number 424 with asparagine and a substitution of lysine at position number 428 with alanine.

43. The host cell according to claim 41, wherein the one or more of the OfCCD1 genes is truncated; optionally wherein the N-terminal fusion partner is selected from the group consisting of small ubiquitin-like modifier protein, maltose binding protein and thioredoxin (trxA), optionally wherein the N-terminal fusion partner is trxA; optionally wherein the one or more of the OfCCD1 and trxA genes is overexpressed.

44. The host cell according to claim 39, wherein the lycopene cyclase gene is a lycopene epsilon-cyclase (LCYe) gene; optionally wherein LCYe gene is isolated from a plant selected from the group consisting of Lactuca sativa, Arabidopsis thaliana, Nicotiana tabacum and Brassica oleracea var. capitata; optionally wherein the LCYe gene isolated from Lactuca sativa is truncated at the N-terminal; optionally wherein the LCYe is truncated from amino acid positions 1 to 50, optionally wherein the truncated LCYe comprises polynucleotide sequence set forth in SEQ ID No: 3.

45. The host cell according to claim 1, wherein the hydroperoxide reductase or the catalase gene is isolated from a prokaryote, optionally wherein the prokaryote is Escherichia coli; optionally wherein the hydroperoxide reductase isolated from Escherichia coli is alkyl hydroperoxide reductase (ahpC/F), wherein the catalase isolated from Escherichia coli is catalase G (KatG); optionally wherein the KatG or ahpC/F is overexpressed.

46. The host cell according to claim 1, wherein the one or more genes are operably linked to one or more inducible promoters, wherein the inducible promoter is a T7 promoter, optionally wherein the T7 promoter is a variant of the wild-type T7 promoter.

47. The host cell according to claim 1, wherein the host cell is modified to not express at least one gene involved in amino acid synthesis; optionally wherein the at least one gene involved in amino acid synthesis is aroA, aroB, aroC and serC; optionally wherein the host cell is a bacterial cell; optionally wherein the bacterial cell is an Escherichia coli cell.

48. A method of producing one or more apocarotenoids comprising culturing the host cell according to claim 1 in a culture medium.

49. The method according to claim 48, wherein the one or more apocarotenoids is selected from the group consisting of α-ionone, β-ionone, psi-ionone, 6-methyl-5-heptene-2-one, hydroxy-ionone, retinol and retinal.

50. The method according to claim 48, wherein the culture medium comprises an inducer and one or more carbon substrates, optionally wherein the culture medium comprises at least one antibiotic; optionally wherein the inducer is IPTG or lactose, wherein the one or more carbon substrate is glucose, glycerol or both, and wherein the at least one antibiotic is selected from the group consisting of ampicillin, chloramphenicol, kanamycin and spectinomycin.

51. The method according claim 48, wherein the host cell is cultured in the culture medium at a shaking speed of between about 50 rpm and about 2000 rpm.

52. The method according to claim 48, wherein the yield of α-ionone in the fed-batch fermentation culture medium is at least 500 mg/L, optionally wherein the yield of α-ionone is at least 700 mg/L.

53. The method according to claim 48, wherein the yield of α-ionone in the batch culture medium in the flask and the fed-batch fermentation culture medium is determined by titer per OD600 (titer/OD600), optionally wherein the titre per OD600 is between about 3-5 mg/L/OD600.

54. A kit for producing one or more apocarotenoids, wherein the kit comprises the host cell according to claim 1 with instructions for use.

55. The kit of claim 54, wherein the host cell is dissolved in solution or lyophilized; optionally wherein the host cell is preserved by deep freezing.


SEQUENCE LISTING (13 sequences)

SEQ ID NO: 1 - protein, 543 aa, Osmanthus fragrans
MGMQGEDAQRTGNIVAVKPKPSQGLTSKAIDWLEWLFVKMMHDSKQPLHYLSGNFAPVDETPPLKDLPVTGHLPECLNGEFVRVGPNPKFASIAGYHWFDGDGMIHGMRIKDGKATYVSRYVQTSRLKQEEFFGRAMFMKIGDLKGMFGLLMVNMQMLRAKLKVLDISYGIGTANTALVYHHGKLLALSEADKPYAIKVLEDGDLQTIGLLDYDKRLAHSFTAHPKVDPFTGEMFTFGYSHTPPYVTYRVISKDGAMNDPVPITVSGPIMMHDFAITENYAIFMDLPLYFKPKEMVKDKKFIFSFDATQKARFGILPRYAKNELLIKWFELPNCFIFHNANAWEEGDEVVLITCRLENPDLDMVNSTVKERLDNFKNELYEMRFNLQNGLASQKKLSVSAVDFPRVNESYTTRKQRYVYGTTLDKIAKVTGIIKFDLHAEPETGKEKLELGGNVKGIFDLGPGRFGSEAVFVPRHPGITSEEDDGYLIFFVHDENTGKSAVNVIDAKTMSPDPVAVVELPKRVPYGFHAFFVTEDQLQEQAKV

SEQ ID NO: 2 - DNA, 1968 bp, Osmanthus fragrans
  source 1..1968: mol_type = other DNA
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcgggtcgtaaagaaagcgacgacggcgttgaacgcatcgaaggtggcgtggttgttgtaaacccaaaaccaaagaaaggcatcactgcatcgaaagcaattgactggctggagtggctgtttgttaaaatgatgcatgacagcaaacagccgctgcactacttgtccggtaacttcgcaccggttgacgaaaccccgccgctgaaagacctgccggtgaccggccatctgccggaatgcctgaacggcgagtttgtgcgtgtcggtccgaacccgaaattcgcttccatcgccggttaccactggtttgacggtgatggtatgatccatggtatgcgcattaaggacggtaaagccacctatgtttctcgttatgttcagacctcccgcctcaaacaggaagaattcttcggtcgtgctatgtttatgaaaattggagatctgaaaggtatgttcggcctgttcaccgtttacatgcagatgctgcgtgcgaaactgaaagtcctggacgcatcttacggtattggtaccgccaacaccgcgctggtttaccaccatggcaaactgctcgctctgagcgaagctgacaaaccgtatgcgatcaaagttctggaagatggcgacctgcagaccatcggcctgttagattacgacaaacgcctggcacatagtttcaccgcacatcccaaggttgacccgtttacaggcgaaatgttcacgttcggttattcccacaccccgccgtatgtgacctaccgtgttatctctaaagatggtgcaatgaacgatccggtacctatcacggtctccggcccaatcatgatgcacgacttcgccatcacggaaaactacgctattttcatggatttgccgctgtacttcaaaccaaaagagatggtgaaagataaaaagttcatcttcagcttcgacgcaacccagaaggctcgctttggcatcctgccgcgctacgccaaaaacgaatctctgatcaaatggttcgagctcccgaactgcttcatctttcacaatgcgaacgcttgggaagagggtgacgaagttgtccttattacctgccgtctggaaaacccggacctggacatggttaacagcaccgttaaagaacgtctggacaacttcaaaaacgaactgtacgaaatgcgcttcaacctgcagaacggtctggctagccagaaaaaactgagcgtaagcgcggttgattttccgcgtgttaacgaatcgtacaccacccgtaaacagcgttatgtttatggtacgaccctggataacatcgcgaaagttaccggtatcatcaaattcgacctgcacgccgaaccggaaacgggcaaagaaaagctggaactgggtggcaacgtaaaaggtatttttgatctgggcccgggtcgcttcggtagtgaagccgttttcgttccacgtcacccgggcatcacctccgaagaagatgacggttatctcatttttttcgtgcatgatgagaacacaggtaaatctgcagtaaacgtgatcgacgcaaaaaccatgtctcctgacccggttgcagtagtcgaactgccgaaacgcgtgccatacggctttcacgcttttttcgtaaccgaagaccagctgcaggaacaggccaaagtgtaa

SEQ ID NO: 3 - DNA, 1452 bp, Lactuca sativa
  source 1..1452: mol_type = other DNA
atgaagtgctccgcaaaatccgaccgttgcgttgttgacaaacaagggattagcgtggctgatgaggaagactacgttaaggcaggcggttccgaacttttctttgtgcagatgcagcgcaccaagtccatggaatcccagtccaaactgtccgaaaaactggcgcagattccgattggcaattgcatccttgacctggtggttattggttgtggcccggcaggtctggcgcttgcggctgagtccgcaaaactgggtctgaacgttggcctcatcggtccggacctcccattcaccaacaactacggcgtttggcaggacgaattcatcggtctcggtctggaaggctgtatcgaacactcctggaaagacaccctggtgtacctggatgatgcggacccaatccgtatcggccgtgcgtacggccgcgtgcaccgtgacctgctgcacgaagaactcctgcgtcgctgtgtggagtctggtgttagctatctgagttccaaggtagagcgtattaccgaagctccaaacggctacagcctgatcgaatgcgaaggtaacatcactatcccgtgtcgtctggcaaccgtggcgtccggcgcggcgagcggtaaattcctggaatacgaactgggcgggccgcgtgtttgcgttcagaccgcttacggtattgaagtagaagttgaaaacaacccgtacgatccggatctgatggtatttatggattaccgtgacttctcaaaacataaaccggaaagcctggaagctaaataccctaccttcctgtatgttatggcgatgtctccgaccaaaatcttcttcgaggagacctgcctggcgtcccgcgaagcaatgccgttcaacctgctgaaaagcaaactgatgagccgtctgaaagccatgggtatccgtattacccgtacctatgaagaagaatggagctatatcccggtaggtggctctctcccgaacacggaacagaaaaacctggcgttcggcgcggctgcatccatggttcacccggctactggctactccgttgttcgttccctgtctgaagccccgaactatgcagctgtaatcgctaaaatcctgcgtcaggatcagtctaaggaaatgatttctctcggtaaatacaccaacatttccaaacaggcgtgggaaaccctctggccgctggaacgcaaacgccagcgtgcatttttcctcttcggtctgtcccacatcgtgctcatggatctggaaggcacccgtaccttcttccgcaccttctttcgcctgccgaaatggatgtggtggggcttcctcggttcctctctgtcttctaccgacctgatcatcttcgctctgtacatgttcgtaatcgcgccgcacagcctgcgcatggaactggttcgtcacctgctgtcggacccgaccggtgctaccatggtaaaagcatacttaactatt

SEQ ID NO: 4 - DNA, 1956 bp, Osmanthus fragrans
  source 1..1956: mol_type = genomic DNA
ttacactttggcctgttcctgcagctggtcttcggttacgaaaaaagcgtgaaagccgtatggcacgcgtttcggcagttcgactactgcaaccgggtcaggagacatggtttttgcgtcgatcacgtttactgcagatttacctgtgttctcatcatgcacgaaaaaaatgagataaccgtcatcttcttcggaggtgatgcccgggtgacgtggaacgaaaacggcttcactaccgaagcgacccgggcccagatcaaaaataccttttacgttgccacccagttccagcttttctttgcccgtttccggttcggcgtgcaggtcgaatttgatgataccggtaactttcgcgattttatccagggtcgtaccataaacataacgctgtttacgggtggtgtacgattcgttaacacgcggaaaatcaaccgcgcttacgctcagttttttctggctagccagaccgttctgcaggttgaagcgcatttcgtacagttcgtttttgaagttgtccagacgttctttaacggtgctgttaaccatgtccaggtccgggttttccagacggcaggtaataaggacaacttcgtcaccctcttcccaagcgttcgcattgtgaaagatgaagcagttcgggagctcgaaccatttgatcagcagttcgtttttggcgtagcgcggcaggatgccaaagcgagccttctgggttgcgtcgaagctgaagatgaactttttatctttcaccatctcttttggtttgaagtacagcggcaaatccatgaaaatagcgtagttttccgtgatggcgaagtcgtgcatcatgattgggccggagaccgtgataggtaccggatcgttcattgcaccatctttagagataacacggtaggtcacatacggcggggtgtgggaataaccgaacgtgaacatttcgcctgtaaacgggtcaaccttgggatgtgcggtgaaactatgtgccaggcgtttgtcgtaatctaacaggccgatggtctgcaggtcgccatcttccagaactttgatcgcatacggtttgtcagcttcgctcagagcgagcagtttgccatggtggtaaaccagcgcggtgttggcggtaccaataccgtaagagatgtccaggactttcagtttcgcacgcagcatctgcatgttaaccatcagcaggccgaacatacctttcagatctccaattttcataaacatagcacgaccgaagaattcttcctgtttgaggcgggaggtctgaacataacgagaaacataggtggctttaccgtccttaatgcgcataccatggatcataccatcaccgtcaaaccagtggtaaccggcgatggaagcgaatttcgggttcggaccgacacgcacaaactcgccgttcaggcattccggcagatggccggtcaccggcaggtctttcagcggcggggtttcgtcaaccggtgcgaagttaccggacaagtagtgcagcggctgtttgctgtcatgcatcattttaacaaacagccactccagccagtcaattgctttcgaagtcagtccctgggaaggtttcggtttcacggcaacaatgttacctgtacgctgtgcgtcttcaccctgcattcccgccaggttagcgtcgaggaactctttcaactgacctttagacagtgcacccactttggttgccgccacttcaccgtttttgaacagcagcagagtcgggataccacggatgccatatttcggcgcagtgccagggttttgatcgatgttcagttttgcaacggtcagtttgccctgatattcgtcagcgatttcatccagaatcggggcgatcattttgcacggaccgcaccactctgcccagaaatcgacgaggatcgccccgtccgctttgagtacatccgtgtcaaaactgtcgtcagtcaggtgaataattttatcgctcat

SEQ ID NO: 5 - DNA, 1780 bp, Lactuca sativa
  source 1..1780: mol_type = genomic DNA
gaaacaaatgacgtgaaagttcttcaaaattgaattaattgtaatcctgaaaacttgatttgtgatagaagaatcaatggagtgctttggagctcgaaacatgacggcaacaatggcggtttttacgtgccctagattcacggactgtaatatcaggcacaaattttcgttactgaaacaacgaagatttactaatttatcagcatcgtcttcgttgcgtcaaattaagtgcagcgctaaaagcgaccgttgtgtagtggataaacaagggatttccgtagcagacgaagaagattatgtgaaggccggtggatcggagctgttttttgttcaaatgcagcggactaagtccatggaaagccagtctaaactttccgaaaagctagcacagataccaattggaaattgcatacttgatctggttgtaatcggttgtggccctgctggccttgctcttgctgcagagtcagccaaactagggttgaacgttggactcattggccctgatcttccttttacaaacaattatggtgtttggcaggatgaatttataggtcttggacttgaaggatgcattgaacattcttggaaagatactcttgtataccttgatgatgctgatcccatccgcataggtcgtgcatatggcagagttcatcgtgatttacttcatgaagagttgttaagaaggtgtgtggaatcaggtgtttcatatctaagctccaaagtagaaagaatcactgaagctccaaatggctatagtctcattgaatgtgaaggcaatatcaccattccatgcaggcttgctactgttgcatcaggggcagcttcagggaaatttctggagtatgaacttgggggtccccgtgtttgtgtccaaacagcttatggtatagaggttgaggttgaaaacaacccctatgatccagatctaatggtgttcatggattatagagacttctcaaaacataaaccggaatctttagaagcaaaatatccgactttcctctatgtcatggccatgtctccaacaaaaatattcttcgaggaaacttgtttagcttcaagagaagccatgcctttcaatcttctaaagtccaaactcatgtcacgattaaaggcaatgggtatccgaataacaagaacgtacgaagaggaatggtcgtatatccccgtaggtggatcgttacctaatacagaacaaaagaatctcgcatttggtgctgcagctagtatggtgcaccctgccacagggtattcagttgttcgatctttgtcagaagctcctaattatgcagcagtcattgctaagattttaagacaagatcaatctaaagagatgatttctcttggaaaatacactaacatttcaaaacaagcatgggaaacattgtggccacttgaaaggaaaagacagcgagccttctttctattcggactatcacacatcgtgctaatggatctagagggaacacgtacatttttccgtactttctttcgtttgcccaaatggatgtggtggggatttttggggtcttctttatcttcaacggatttgataatatttgcgctttatatgtttgtgatagcacctcacagcttgagaatggaactggttagacatctactttctgatccgacaggggcaactatggtaaaagcatatctcactatatagatttagattatataaataatacccatatcttgcatatatataagccttatttatttcttttgtatccttacaacaacatactcgttaattatatgtttttta

SEQ ID NO: 6 - DNA, 20 bp, synthetic construct
  source 1..20: mol_type = other DNA
gtttgctgccaccgctgagc

SEQ ID NO: 7 - DNA, 20 bp, synthetic construct
  source 1..20: mol_type = other DNA
ttacactttggcctgttcct

SEQ ID NO: 8 - DNA, 40 bp, synthetic construct
  source 1..40: mol_type = other DNA
aggaacaggccaaagtgtaagtaaggtaaaacttatcgat

SEQ ID NO: 9 - DNA, 40 bp, synthetic construct
  source 1..40: mol_type = other DNA
gctcagcggtggcagcaaacttatgcagttttggtgcgaa

SEQ ID NO: 10 - DNA, 40 bp, synthetic construct
  source 1..40: mol_type = other DNA
aggaacaggccaaagtgtaaccaacaatatgtaagatctc

SEQ ID NO: 11 - DNA, 40 bp, synthetic construct
  source 1..40: mol_type = other DNA
gctcagcggtggcagcaaacttacagcaggtcgaaacggt

SEQ ID NO: 12 - DNA, 22 bp, synthetic construct
  source 1..22: mol_type = other DNA
ccggttgcagtagtcgaactgc

SEQ ID NO: 13 - DNA, 21 bp, synthetic construct
  source 1..21: mol_type = other DNA
gccggtactgccgggcctctt